(19) United States
(12) Patent Application Publication
(10) Pub. No.: US 2021/0145728 A1
(43) Pub. Date: May 20, 2021

(54) COMPOSITION DERIVED FROM SEAWATER, METHOD FOR OBTAINING SAME AND USE THEREOF IN COSMETIC COMPOSITIONS

(71) Applicant: Laboratoires Gilbert, Herouville Saint-Clair (FR)

(72) Inventors: Sylvie CHOPIN, Langrune-Sur-Mer (FR); Séverine Sylvie Eliane PLANTIER, Brest (FR)

(21) Appl. No.: 17/046,572

(22) PCT Filed: Apr. 11, 2019
(86) PCT No.: PCT/EP2019/059259

§ 371 Date: Oct. 9, 2020

(30) Foreign Application Priority Data
Apr. 12, 2018 (FR) 1853214

(51) Int. Cl. A61K 8/96; A61K 8/19; A61Q 19/00; A61Q 19/10

(52) U.S. Cl. CPC A61K 8/965; A61K 8/19; A61K 2800/10; A61Q 19/10; A61K 2800/83; A61Q 19/007

(57) ABSTRACT

A composition derived from seawater enriched in magnesium and depleted in potassium, the method for preparing the composition and its use as a moisturising and/or soothing cosmetic agent. A cosmetic composition includes the composition derived from seawater and a cosmetic treatment method includes the topical application of the composition.

### TECHNICAL AREA

The present invention relates to cosmetic compositions based on natural active substances.

### STATE OF THE ART

Seawater is commonly used in compositions for nasal and ear hygiene. These compositions, generally of the spray type or in single doses, may contain only seawater. This seawater has been filtered beforehand to remove the impurities and has generally been objected to an electrodialysis step, in order to reduce the content in salts which may be irritating to the mucous membranes in high concentrations.

Furthermore, application WO2006/000455 describes a bioactive composition obtained from seawater which is suitable for the hygiene of the female genital sphere and helps to tone the musculoligamentous structure which supports the entire genital arrangement. Compared to natural seawater, this composition is enriched in divalent calcium and magnesium ions and is reduced in monovalent sodium and potassium ions. The method for preparing this composition comprises a step of selective demineralization of seawater by electrodialysis, the acidification of the solution thus obtained by dissociation of the water through ion exchange membranes and the concentration of the solution. thus obtained by nanofiltration. Document WO2006/000455 more particularly describes a composition comprising 3500 mg/l of sodium chloride (NaCl), 1850 mg/l of magnesium Mg²⁺), 600 mg/l of calcium (Ca²⁺) and 325 mg/l of potassium (K+) and a composition comprising 3500 mg/l of sodium chloride, 2200 mg/l of magnesium, 530 mg/l of calcium and 325 mg/l of potassium.

In the cosmetics field, it is useful to have available multifunctional agents for the preparation of cosmetic compositions, which makes it possible to reduce the number of ingredients to be used in these compositions. These multifunctional agents are, for example, used both as an active cosmetic agent and as a texturizing agent, excipient, colorant and/or preservative. In addition, cosmetic products based on ingredients of natural origin are very popular with consumers.

There is still a need to provide new natural active substances having a cosmetic and multifunctional effect.

### DETAILED DESCRIPTION

The inventors have demonstrated, surprisingly, a composition derived from seawater having advantageous cosmetic properties. This composition derived from seawater in fact has moisturizing and/or soothing properties, while being a cosmetic excipient. This composition derived from seawater may therefore be advantageously used as a multifunctional natural active substance in cosmetic compositions.

More particularly, the inventors have demonstrated a new composition derived from seawater comprising potassium at a concentration less than or equal to 100 mg/l, and magnesium at a concentration greater than or equal to 1600 mg/l. Advantageously, the composition derived from seawater has a sodium concentration less than or equal to 1800 mg/l and a calcium concentration greater than or equal to 460 mg/l.

This composition derived from seawater is obtained by an improved preparation method, comprising a selective electrodialysis step and a nanofiltration step. Surprisingly, the volume yield of the method according to the invention is indeed improved, in particular thanks to the improved volume yield of the electrodialysis step, even while the electrodialysis step is prolonged. The volume yield of the electrodialysis step is, in particular, greater than 85%. However, an increase in the volume yield of a method of only 4% is sufficient to provide a substantial improvement, in particular when the method is implemented on an industrial scale.

A first object of the invention is a composition derived from seawater, characterized in that it comprises potassium at a concentration less than or equal to 100 mg/l and magnesium at a concentration greater than or equal to 1600 mg/l.

Advantageously, the composition derived from seawater as defined above comprises magnesium at a concentration of 1600 mg/l to 3000 mg/l.

The composition derived from seawater as defined above is preferably characterized in that it is obtained by:
a step of selective electrodialysis of a sample of seawater, to obtain a solution depleted in monovalent ions, and
a step of concentrating said solution by nanofiltration, to obtain a solution enriched in divalent ions.

The composition derived from seawater as defined above is preferably characterized in that it has an osmotic concentration greater than or equal to 320 mOsm/l.

The composition derived from seawater as defined above preferably has a pH of less than 6.5.

The composition derived from seawater as defined above is preferably hypertonic.

The composition derived from seawater as defined above preferably comprises:
sodium at a concentration less than or equal to 1800 mg/l, and/or
calcium at a concentration greater than or equal to 460 mg/l.

Another object of the invention is a composition, preferably a cosmetic composition, comprising a composition derived from seawater as defined above.

Said composition preferably comprises at least 2% of said composition derived from seawater, the percentage being expressed in g per 100 g of composition. Said composition may also consist of a composition derived from seawater.

Another object of the invention is a device comprising a composition derived from seawater as defined above, preferably a cosmetic composition as defined above.

Another object of the invention is a method for obtaining a composition derived from seawater as defined above, characterized in that said method comprises:
a step of selective electrodialysis of a sample of seawater, optionally filtered, until a solution is obtained with a conductivity greater than or equal to 15 mS/cm and less than or equal to 18 mS/cm, and
a step of nanofiltration of said solution.

Another object of the invention relates to the use of a composition derived from seawater as defined above as a cosmetic ingredient. An object of the present invention is, for example, the use of a composition derived from seawater as a moisturizing cosmetic agent and/or as a soothing cosmetic agent, the composition derived from seawater preferably being a composition such as defined above or obtained by the method as defined above.

Another object of the invention is a method for preparing a cosmetic composition, characterized in that said method comprises a step of mixing a composition derived from seawater as defined above or obtained by the method as defined above with at least one other cosmetic ingredient.

Another object of the invention relates to a cosmetic treatment method, characterized in that it comprises the topical application of a cosmetic composition as defined above or obtained by the method as defined above.

Composition Derived from Seawater

The present invention relates to a composition derived from seawater.

The expression “composition derived from seawater” or “composition based on seawater” as used herein means a composition obtained from seawater.

In particular, a composition derived from seawater is obtained by at least one seawater treatment step which makes it possible to modify its initial composition, preferably by increasing its concentration in magnesium and/or in calcium and by reducing its sodium and potassium concentration.

The term “magnesium” refers here to the magnesium ion (Mg²⁺). The term “calcium” refers here to the calcium ion (Ca²⁺).

By the term “potassium” is meant here the potassium ion (K⁺).

By the term “sodium” is meant here the sodium ion (Na⁺).

By the term “chloride” is meant here the chloride ion (Cl—).

The concentration of a given ion is preferably measured by Inductively Coupled Plasma Mass Spectrometry, or ICP-MS.

A composition derived from seawater according to the invention is therefore not a composition obtained by a simple step of diluting seawater or evaporating seawater.

A composition derived from seawater according to the invention is preferably obtained by a method not comprising a step of diluting seawater and/or not comprising a step of evaporating the water from sea.

An example of a seawater treatment step modifying its initial composition is a filtration step or an electrodialysis step, in particular selective electrodialysis.

Said filtration step may be a microfiltration, ultrafiltration or nanofiltration step.

Microfiltration, ultrafiltration and nanofiltration are techniques for the physical separation of elements contained in a liquid.

Microfiltration uses filter membranes with pore diameter between 0.1 μm and 10 μm. Microfiltration, for example, eliminates suspended matter, microalgae and bacteria present in a liquid.

Ultrafiltration uses semi-permeable membranes whose pore diameter is between 0.001 μm and 0.1 μm.

Nanofiltration uses membranes with a pore diameter of less than 0.001 μm.

The seawater used to obtain a composition derived from seawater preferably has the following characteristics:
a salinity of 30 g/I to 35 g/I,
a conductivity of 40 mS/cm to 50 mS/cm,
a pH of 6.8 to 8.5,
a sodium concentration of 10,000 mg/l to 12,000 mg/l,
a chloride concentration of 17,000 mg/l to 22,000 mg/l,
a potassium concentration of 350 mg/l to 450 mg/l,
a magnesium concentration of 1200 mg/l to 1600 mg/l, and
a calcium concentration of 390 mg/l to 480 mg/l.

Salinity refers to the content of salts dissolved in a liquid composition. For example, the salinity of an aqueous composition is the sum of the ion concentrations present in the water.

The composition derived from seawater is preferably obtained from seawater by at least one of the following steps, preferably at least two, more preferably the following three steps:
a microfiltration step,
an electrodialysis step, in particular selective electrodialysis,
a nanofiltration step.

The composition derived from seawater is preferably obtained by:
a step of selective electrodialysis of a sample of seawater, to obtain a solution depleted in monovalent ions, preferably while retaining the contents of the divalent Mg²⁺ and Ca²⁺ ions present in seawater, and
a step of concentration of said solution by nanofiltration, to obtain a solution concentrated in divalent ions, preferably to obtain a concentration factor of divalent ions relative to seawater of 1.3 to 1.6.

The electrodialysis step is advantageously carried out until a solution is obtained having a conductivity of from 15 mS/cm to 18 mS/cm, for example from 16 mS/cm to 17 mS/cm.

The nanofiltration concentration step is advantageously carried out until a solution is obtained having a conductivity of from 20 mS/cm to 23 mS/cm, for example from 20 mS/cm to 21 mS/cm.

The conductivity measurement is preferably carried out at 20° C.

The selective electrodialysis step preferably makes it possible to obtain a solution depleted in monovalent ions, with a concentration for the other ions equivalent to that present in the seawater sample.

Advantageously, the composition derived from seawater according to the invention does not include elements other than those naturally present in seawater or possibly provided by the brine solution during the electrodialysis step.

A preferred composition derived from seawater is a composition as defined above comprising:
potassium at a concentration less than or equal to 100 mg/l, preferably less than or equal to 90 mg/l, preferably less than 80 mg/l, preferably less than 70 mg/l, more preferably less than 60 mg/l, and
magnesium at a concentration greater than or equal to 1600 mg/l, preferably greater than or equal to 1700 mg/l, more preferably greater than or equal to 1750 mg/l.

The composition derived from seawater is preferably a composition as defined above, characterized in that it has an osmotic concentration:
(i) greater than or equal to 320 mOsm/l, preferably greater than or equal to 330 mOsm/l, more preferably greater than or equal to 340 mOsm/l, for example greater than or equal to 350 mOsm/l, more preferably still greater or equal to 360 mOsm/l, and/or
(ii) less than or equal to 480 mOsm/l, preferably less than or equal to 460 mOsm/l, preferably less than or equal to 440 mOsm/l, more preferably less than or equal to 430 mOsm/l.

The composition derived from seawater has, for example, an osmotic concentration of from 320 mOsm/l to 410 mOsm/l, preferably from 320 mOsm/l to 390 mOsm/l, more preferably from 330 mOsm/l to 370 mOsm/l.

By “osmotic concentration”, also called “osmolarity”, is meant the number of osmotically active particles per liter of solution.

The term “mOsm/L” is an abbreviation for milli-osmole per liter.

The composition derived from seawater is preferably a composition as defined above comprising:
sodium at a concentration less than or equal to 1800 mg/l, more preferably less than or equal to 1750 mg/l, more preferably less than or equal to 1700 mg/l, more preferably still less than or equal to 1650 mg/l and/or
calcium at a concentration greater than that of seawater, preferably greater than or equal to 460 mg/l, preferably greater than or equal to 490 mg/l, more preferably greater than or equal to 520 mg/l, more preferably greater than or equal to 550 mg/l.

The composition derived from seawater is a composition as defined above comprising, preferably, magnesium at a concentration of less than 3000 mg/l.

In a preferred embodiment, the composition derived from seawater is a composition as defined above, comprising:
potassium at a concentration of 20 mg/l to 100 mg/l, preferably 20 mg/l to 80 mg/l, preferably 30 mg/l to 70 mg/l, more preferably 40 to 60 mg/l,
magnesium at a concentration of 1600 mg/l to 2100 mg/, preferably from 1630 g/to 2000 mg/l, preferably from 1660 mg/l to 1900 mg/l, more preferably from 1700 to 1800 mg/l,
sodium at a concentration of 1300 mg/l to 1800 mg/l, preferably from 1500 mg/l to 1700 mg/l, more preferably from 1600 mg/l to 1650 mg/l, and/or
calcium at a concentration of 460 mg/l to 750 mg/l, preferably from 500 mg/to 700 mg/l, preferably from 530 mg/l to 650 mg/l, more preferably from 550 to 600 mg/l.

The composition derived from seawater as defined above preferably comprises chloride ions (Cl⁻) at a concentration of 6000 mg/l and 9200 mg/l, preferably of 6300 mg/l and 9000 mg/l, more preferably 6500 mg/l and 8800 mg/l.

The composition derived from seawater according to the invention is therefore a liquid composition.

The composition derived from seawater is preferably hypertonic.

A “hypertonic composition” here denotes a composition whose salinity is greater than or equal to 9.5 g/I, preferably greater than or equal to 10 g/I.

The composition derived from seawater is for example obtained by a preparation method as defined below.

A composition derived from seawater has the advantage of containing the elements initially present in seawater. However, more than two thirds of the 94 natural chemical elements are present in seawater. The composition derived from seawater therefore has a very different composition from thermal waters for example. Furthermore, thermal waters are particularly poor in chloride, magnesium and copper compared to the composition derived from seawater according to the invention and have a neutral to basic pH.

The composition derived from seawater as defined above preferably has a pH less than or equal to 6.5, more preferably less than or equal to 6, for example less than or equal to 5.5 or less than or equal at 5.

For example, the composition derived from seawater as defined above may have a pH of 4.5 to 6.5, preferably 5 to 6 or 4.5 to 5. This acidic pH is particularly suitable for cosmetic use, allowing the skin pH to be respected.

Method for Preparing a Composition Derived from Seawater

An object of the present invention is also a method for preparing a composition derived from seawater, in particular as defined above.

The method according to the invention comprises, in particular, an electrodialysis step, in particular selective electrodialysis, and a nanofiltration step.

The method may advantageously comprise a preliminary step of filtering the seawater (also called “pre-filtration” hereinafter).

Seawater Pre-Filtration

The seawater is preferably filtered beforehand, in order to obtain clean and/or sterile water. The pre-filtration step, by at least partially eliminating the organic matter present in the seawater, prevents too rapid clogging of the electrodialysis membranes.

The seawater pre-filtration step is advantageously a microfiltration step.

Preferably, the pre-filtration step comprises several cascade filtration steps, on membranes comprising increasingly smaller pores.

For example, the pre-filtration step may use at least one of the following membranes: a membrane making it possible to remove particles of size greater than 50 μm, a membrane making it possible to eliminate particles of size greater than a size of 20 μm at 50 μm, a membrane making it possible to remove particles of size greater than a size of 10 μm to 20 μm, a membrane making it possible to eliminate particles of size greater than 0.2 μm.

Electrodialysis Step

The electrodialysis step of a sample of seawater, optionally filtered, makes it possible to obtain a solution depleted in monovalent ions.

The electrodialysis is therefore a selective electrodialysis, i.e. carried out using at least one selective membrane, preferably a selective cationic membrane.

The particular object of the electrodialysis step is to desalinate, at least partially, seawater, mostly eliminating the Na⁺ and Cl⁻, ions, but almost completely retaining the Mg²⁺et Ca²⁺ ion contents.

For example, the percentage of ion losses at the end of the electrodialysis step is:
greater than or equal to 80% for sodium (Na⁺),
greater than or equal to 65% for chloride (Cl⁻),
less than or equal to 15% for magnesium (Mg²⁺), and
less than or equal to 15% for calcium (Ca²⁺).

The electrodialysis step also advantageously removes potassium K+ ions. The percentage of potassium ion losses at the end of the electrodialysis step is, for example, greater than 85%.

The seawater, preferably pre-filtered, is introduced into a compartment called a diluate.

The method for preparing a composition derived from seawater according to the invention, therefore does not include a step of evaporating the seawater. The seawater used in the electrodialysis step is thus not a brine.

The electrodialysis is preferably carried out in a closed circuit. This means that at the end of the electrodialysis, the at least partially desalinated seawater is reintroduced into the diluate compartment.

The membranes used for the electrodialysis step include at least one cationic membrane and at least one anionic membrane.

The cationic membrane is, for example a membrane of the CMX type.

The cationic membrane is preferably selective. A selective cationic membrane allows monovalent ions to pass, but the divalent ions to be retained, in order to limit the loss of divalent ions during the electrodialysis.

The anionic membrane is for example of the AMX type.

The electrodialysis step may be carried out, for example, using 20 to 80 membranes, for example 50 membranes to 80 membranes, for example 50, 55, 60, 65, 70, 75 or 80 membranes.

The flow rate of the eluate (corresponding to seawater) may be, for example, from 5001/h to 10001/h, preferably from 600 l/h to 10001/h.

The flow rate of the concentrate (also called brine) may be, for example, from 5001/h to 10001/h, preferably from 6001/h to 10001/h.

The flow rate for the electrolyte solution may be, for example from 1001/h to 4001/h, preferably from 1501/h to 4001/h.

The electrodialysis step is preferably carried out at constant pressure.

The electrolyte solution may be, for example, a solution of KNO₃ or NaCl, preferably KNO₃.

The conductivity of the electrolyte solution is for example 20 mS/cm.

The brine solution may be, for example, an NaCl solution adjusted to pH 1.6 by adding HCl.

The voltage during the electrodialysis step is preferably greater than or equal to 45V, more preferably greater than or equal to 50V.

The voltage during the electrodialysis step may be, for example, from 45V to 80V, preferably from 50V to 80V, more preferably from 60V to 80V.

The conductivity of the brine solution is, for example, from 8 mS/cm to 12 mS/cm, preferably 10 mS/cm.

The electrodialysis is stopped once the desired conductivity is reached.

In particular, the electrodialysis is stopped once the solution has a conductivity greater than or equal to 15 mS/cm and/or less than or equal to 18 mS/cm. In an advantageous embodiment, the electrodialysis step is carried out until a solution is obtained having a conductivity of 15 mS/cm to 18 mS/cm, preferably 16 to 17 mS/cm.

The conductivity is preferably measured at 20° C.

The duration of the electrodialysis step is longer than in the methods usually used. In particular, the electrodialysis step is carried out for a period greater than or equal to 3 hours, for example greater than or equal to 4 hours. Preferably, the electrodialysis step is carried out for a duration greater than or equal to 3 hours, for example greater than or equal to 4 hours, and/or for a duration less than or equal to 6 hours, for example less than or equal to 5 hours.

Conductivity (also called “sigma” or “σ”) is a physical quantity characterizing the conduction capacity of a substance. In the international system (SI), conductivity is for example expressed in Siemens (S) per meter (m). The conductivity is measured in particular by a conductimeter on a product tempered at 20° C.

The performance of the electrodialysis step is evaluated by the percentage of ion losses in seawater after passing through the electrodialysis.

Advantageously, the electrodialysis step according to the invention allows:
a loss of sodium ions of at least 70%, preferably at least 75%, more preferably at least 80%, for example 85% or at least 85%,
a loss of chloride ions of at least 55%, preferably at least 60%, more preferably at least 65%, for example at least 70%,
a loss of magnesium ions of less than 20%, preferably less than 15%, for example less than 10%,
a loss of calcium ions of less than 20%, preferably less than 15%, for example less than 10%,
a loss of potassium ions greater than 80%, preferably greater than 85%, for example greater than or equal to 89%, and/or
a loss of sulfate ions of less than 20%, preferably less than 15%, for example less than 10%;

the loss being expressed relative to the concentration of the starting seawater.

Advantageously, the electrodialysis step according to the invention has a volume yield of at least 85%, preferably at least 90%, for example 91%.

Nanofiltration Step

The nanofiltration step corresponds to a concentration step. Its main purpose is to increase the concentration of divalent cations.

Nanofiltration is performed on the solution obtained at the end of the electrodialysis step.

The nanofiltration step is preferably carried out in a closed circuit. This means that the concentrate obtained at the end of the nanofiltration is reintroduced into the nanofiltration feed tank.

The flow rate may be, for example, from 3001/h to 12001/h, preferably from 3201/h to 11001/h, more preferably from 3301/h to 13001/h.

The flow rate of the retentate may be, for example, from 2001/h to 9001/h, preferably from 2501/h to 8001/h, more preferably from 2701/h to 7001/h.

The permeate flow may be, for example, from 101/h/m² to 1801/h/m², Preferably from 151/h/m² to 1601/h/m², more preferably from 201/h/m²/m² to 1401/h/m².

The transmembrane pressure during the nanofiltration step is, for example, from 5 to 15 bar, preferably from 7 to 12 bar, for example 10 bar.

The nanofiltration step is carried out, for example, on a polyamide membrane.

The membrane used for the nanofiltration step may be a selective membrane or not.

When the membrane used for the nanofiltration step is not a selective membrane, it must have a good retention rate of divalent ions, for example greater than 97%.

The nanofiltration step is preferably carried out until the desired concentration factor is obtained, in particular of magnesium and calcium. The magnesium and/or calcium concentration factor is preferably greater than or equal to 1.3, for example greater than or equal to 1.4, relative to the solution resulting from the electrodialysis.

The magnesium and/or calcium concentration factor is, for example, from 1.4 to 2, more preferably from 1.4 to 1.6, relative to the solution resulting from the electrodialysis.

The nanofiltration step is at least 1 h, preferably 1 h30 or at least 1 h30.

The conductivity of the solution obtained at the end of the nanofiltration is preferably from 20 to 23 mS/cm, more preferably from 20 to 21 mS/cm.

The nanofiltration step is preferably carried out until a solution is obtained having a conductivity of 20 to 23 mS/cm, for example 20 to 21 mS/cm.

The conductivity is preferably measured on a conductivity meter and a solution at 20° C.

The volume yield of the nanofiltration step is preferably greater than or equal to 55%, more preferably greater than or equal to 58%, more preferably greater than or equal to 60%.

The volume yield of the method according to the invention is preferably greater than or equal to 50%, preferably greater than or equal to 51%, preferably greater than or equal to 52%, more preferably greater than or equal to 53%, more preferably still greater or equal to 54% or 55%.

Composition Comprising a Composition Derived from Seawater

An object of the present invention is also a composition comprising a composition derived from seawater as defined above.

In one embodiment, the composition may comprise at least 0.1% of composition derived from seawater, preferably at least 1%, preferably at least 2%, the percentage being expressed by mass over the total mass. of the composition.

In another embodiment, the composition may comprise at least 5% of composition derived from seawater, preferably at least 8%, more preferably at least 10% of composition derived from seawater, the percentage being expressed by mass over the total mass of the composition.

In yet another embodiment, the composition may comprise at least 15%, at least 20%, at least 25%, at least 30%, at least 35%, at least 40%, at least 45%, more preferably still at least 50% of composition derived from seawater, the percentage being expressed by mass over the total mass of the composition.

In yet another embodiment, the composition may comprise at least 55%, at least 60%, at least 65%, at least 70%, at least 75%, at least 80%, at least 85%, more preferably still at least 90% of composition derived from seawater, the percentage being expressed by mass over the total mass of the composition.

A preferred composition comprises for example from 1% to 99%, preferably from 1.5% to 99%, preferably from 2% to 99%, more preferably from 2% to 90%, more preferably from 2% to 80%, more preferably from 2% to 70%, more preferably from 2% to 60%, more preferably from 2% to 50% of composition derived from seawater, the percentage being expressed by mass over the total mass of the composition.

A preferred composition may comprise, for example, from 2% to 10%, from 10% to 20%, from 20% to 30%, from 30% to 40% or from 40% to 50% of composition derived from seawater, the percentage being expressed by mass over the total mass of the composition.

A preferred composition comprises, for example, 2%, 10%, 50% or 100% of composition derived from seafood, the percentage being expressed by mass over the total mass of the composition.

In a preferred embodiment, the composition comprising a composition derived from seawater as defined above is a cosmetic composition.

Cosmetic Composition

Since the composition derived from seawater has moisturizing and soothing properties, it may be used advantageously as a cosmetic ingredient in a cosmetic composition, for example both as an active ingredient and as an excipient.

An object of the present invention is thus also a cosmetic composition comprising a composition derived from seawater.

The cosmetic composition may consist of a composition derived from seawater. This may be, for example, a mist. The cosmetic composition is then supplied in a mist.

The composition derived from seawater is in particular as defined above in the section of the same name, for example as obtained by the method for preparing a cosmetic composition derived from seawater as defined above. above.

The concentration of composition derived from seawater in the cosmetic composition is in particular as defined above in the section “Composition comprising a composition derived from seawater”.

In an advantageous embodiment, the cosmetic composition comprises at least one other cosmetic ingredient.

Said other cosmetic ingredient may be an active ingredient and/or an excipient.

The excipient is, for example, selected from the group consisting of a solvent, emulsifier, a stabilizing agent (for example a stabilizing agent of an emulsion), a gelling agent, a surfactant, an emollient, a preservative, a buffering agent, an agent. antioxidant, humectant, surfactant, viscosity control agent, antistatic agent, chelating agent, masking agent, film-forming agent, opacifier, bulking agent, foaming agent, dye, perfume and combinations thereof.

The solvent is, for example, water and/or propanediol.

Said other active ingredient may for example be a skin conditioning agent, a cleansing agent, a soothing agent and/or a skin conditioning agent.

The excipient(s) and/or other active ingredient(s) of the cosmetic composition may be chosen, for example, from the excipients and active ingredients described in the tables of Example 5.

Preferably, the other cosmetic ingredient is not arginine or one of its salts or esters, or more generally is not a basic amino acid or one of its salts or esters.

In a preferred embodiment, the cosmetic composition comprises:
at least one excipient, preferably at least two excipients, more preferably at least three excipients, and
optionally, at least one other active ingredient.

The cosmetic composition is preferably suitable for topical application, in particular as defined below in the section “Cosmetic treatment method”.

The cosmetic composition can be a lotion, a shower gel (preferably a superfat shower gel or a micellar shower gel), a shampoo (preferably an extra mild shampoo), a soap (preferably an extra soft superfat bar), a washing cream, a washing oil (preferably lipid-replenishing), an intimate hygiene washing treatment, a balm, a cream gel, a sunscreen composition, such as a sunscreen, a lotion, a mist or a mask.

The term “superfat” denotes a composition enriched with superfatting agents, such as vegetable oils, vegetable butters, glycerin, agents of animal origin such as lanolin or their combinations.

The cosmetic composition can be in liquid, semi-liquid, solid, powder, micellar, gel or cream form.

The cosmetic compositions preferably have a pH compatible with topical use on the skin or integuments or for use on the genital mucous membranes.

In a particular embodiment, the cosmetic composition as defined above has a pH of 4.5 to 6.5, in particular for application to the skin and/or integuments, or a pH of 3, 5 to 5.5, in particular for application to the genital mucous membranes.

Device Comprising a Composition Derived from Seawater

An object of the present invention is also a device comprising a composition derived from seawater as defined above, a composition as defined above and/or a cosmetic composition as defined above.

The device may be selected, for example, from the group consisting of a misting system, a bottle, an ampoule, a tube, a jar, a spray, a single-dose container or even a wipe, said wipe being impregnated with the composition derived from seawater.

A misting system is for example a mist or a spray bottle.

Method for Preparing a Cosmetic Composition

An object of the present invention is also a method for preparing a cosmetic composition as defined above.

The method then comprises a step of mixing a composition derived from seawater with at least one other cosmetic ingredient.

The composition derived from seawater is in particular as defined above in the section of the same name, for example as obtained in the method for preparing a cosmetic composition derived from seawater as defined above.

The other cosmetic ingredient is, in particular, as defined above in the “Cosmetic composition” section.

Cosmetic Use of a Composition Derived from Seawater

An object of the present invention is also the cosmetic use of a composition derived from seawater, in particular in a cosmetic composition.

By the expression “cosmetic use” is meant a non-therapeutic cosmetic use.

The composition derived from seawater is, in particular, as defined above in the section of the same name, for example as obtained in the method for preparing a composition derived from seawater as defined above.

A more particular object of the present invention is the use of a composition derived from seawater, in particular as defined above, as a moisturizing agent and/or soothing cosmetic agent, in particular in a cosmetic composition.

By “moisturizing cosmetic agent” is meant here an agent which increases the percentage change in the electrical capacity of the epidermis during the time of the study, compared to the initial value.

The moisturizing effect of the composition derived from seawater may be demonstrated by a study of corneometry, as illustrated in Example 3. Corneometry is a method based on the existing relationship between the electrical properties of tissues and their water content.

By “soothing cosmetic agent” is meant here an agent which decreases the undesirable sensations caused by a chemical or physical stimulus.

The soothing effect of the composition derived from seawater may be demonstrated, as illustrated below in Example 2.

The composition derived from seawater also helps maintain the barrier function of the epidermis.

Cosmetic Treatment Method

An object of the present invention is also a cosmetic treatment method, characterized in that it comprises the topical application of a cosmetic composition as defined above.

By the expression “cosmetic treatment method” is meant here a non-therapeutic cosmetic treatment method.

By the expression “topical application” is meant the application to the skin and/or the integuments and/or the mucous membranes.

The skin encompasses the scalp.

The term “integuments” here denote the hair and/or the nails.

Mucous membranes include the oral, nasal, and/or genital mucous membranes. The mucous membranes are preferably the genital mucous membranes.

In a specific embodiment of the invention, topical application refers to application to the skin and/or integuments, excluding the genital mucous membranes, or more generally excluding the mucous membranes.

The cosmetic treatment method is preferably used in humans.

By the expression “in humans” is meant herein a human subject (also called an individual), for example a man or a woman, of any age, for example infants, children, adolescents, adults and the elderly.

In a preferred embodiment, the individual is not suffering from and/or is not likely to suffer from a dermatological disease.

As the composition derived from seawater has a moisturizing and/or soothing effect, the present invention particularly relates to a cosmetic treatment method as defined above in an individual, for example, with dry to very dry skin, sensitive skin with redness, skin presenting sensations of cutaneous discomfort and/or skin with tendencies to atopy or prone to allergies.

A feeling of skin discomfort is for example characterized by tightness, tingling and/or heating.

Dry to very dry skin, sensitive skin with redness, skin with sensations of skin discomfort and/or skin with tendencies to atopy or prone to the allergies mentioned above are non-pathological skin.

Other characteristics and advantages of the invention will emerge more clearly from the examples which follow, given by way of illustration and without limitation.

### EXAMPLES

### Example 1: Method for Preparing a Composition Derived from Seawater

Material and Methods

(i) Electrodialysis (ED)

The electrodialysis reactor comprises an alternation of CMX-type anion-selective (MEA) and CMS-type cation (MEC) membranes with a membrane surface area of 1 dm².

350l of raw seawater pre-filtered at 10 μm are introduced into a compartment called “Diluate” connected to the 50 product compartments of an electrodialysis reactor. 20 L of brine, prepared with purified water, sodium chloride (NaCl) and 0.1N hydrochloric acid (HCl) to achieve a conductivity of 10 mS/cm and a pH of 1.6 are introduced in a tank connected to the 50 brine compartments of the electrodialysis reactor.

20 L of electrolyte prepared with purified water and potassium nitrate (KNO₃) to achieve a conductivity of 20 mS/cm are introduced into a tank connected to the anode and cathode compartments of the electrodialysis reactor.

The product and the brine are circulated at a flow rate of 10001/h (liters/hour) and the electrolyte at a flow rate of 3601/h, using the 3 pumps corresponding to each of these 3 circuits connected to the electrodialysis reactor and simultaneously powered up to 45V across the anode and cathode of the reactor.

The conductivity and pH of the product compartment solution are measured during the electrodialysis.

The electrodialysis is stopped when the conductivity reaches 17 mS/cm.

(ii) Nanofiltration (NF)

The cation concentration of the solution obtained at the end of the electrodialysis is then carried out by passing said solution in a loop through the circuit of a nanofiltration membrane. The concentrate obtained at the outlet of the nanofiltration is thus returned to the feed tank.

The membrane used is an NF270 membrane (Dow filmtec) with a membrane surface area of 2.6 m². The feed flow rate is 350 l/h and that of the retentate is 210 l/h, i.e. a permeate flow of 34 l/h/m² (Liters/hour/square meter).

The conductivity and the pH of the solution are measured during the nanofiltration.

The nanofiltration is stopped when: the conductivity reaches 21 mS/cm and/or when the magnesium concentration is increased by a factor of 1.4 to 1.6 compared to the solution obtained by electrodialysis.

Results

The conductivity of the product compartment solution decreases in proportion to the demineralization exerted from 54 mS/cm to 17 mS/cm and the time that passes. The pH of the solution decreases from 7.4 to 3.7 at the same time. The transfer of the ions is accompanied by a transfer of water from the product tank which loses 301 of its initial volume to the brine tank. The yield of the electrodialysis step is thus 91%. The duration of the electrodialysis step is 296 min and 218 min.

During the nanofiltration step, all the salts and trace elements, due to the extraction in the solution of the permeate consisting of practically pure water (see Tables 1 and 2). The conductivity of the product compartment increases in proportion to the concentration of the solution from 17 mS/cm to 21 mS/cm, while the pH remains almost constant (pH ranging from 3.8 to 4.1).

The volume of the concentrated solution is reduced by 1201 to finally obtain 1921 of a 12 g/l hypertonic solution of all salts and trace elements combined.

The characteristics of the entire method are summarized in Tables 1 and 2 below.

The sodium, magnesium, calcium and potassium concentrations of this solution are as follows: 1610 mg/l (Na⁺), 1770 mg/l (Mg²⁺), 575 mg/l (Ca²⁺) and 51 mg/l (K⁺) (Analyzes carried out by ICP-MS according to NF EN ISO 17294-2) (cf. Table 3 below). The osmotic concentration of the composition thus obtained is 339 to 345 mOsm/l.

The volume yield of the method for obtaining the composition derived from seawater is therefore 55%.

TABLE 1
Evolution of ion concentrations during the method

| | Sea- | After | After | Loss/Gain | Loss/Gain | Total |
|---|---|---|---|---|---|---|
| | water | ED | NF | after ED | after NF | Loss/Gain |
| Na⁺ | 10900 | 1600 | 1610 | −85% | 1% | −85% |
| K⁺ | 420 | 47.5 | 50.5 | −89% | 6% | −88% |
| Mg²⁺ | 1447 | 1300 | 1770 | −10% | 36% | +22% |
| Ca²⁺ | 447 | 420 | 575 | −6% | 37% | +29% |
| Cl⁻ | 20400 | 5825 | 6780 | −71% | 16% | −67% |
| SO₄²⁻ | 2760 | 2515 | 2351 | −9% | −7% | −15% |

TABLE 2
Characteristics of the method for obtaining
a composition derived from seawater

| | |
|---|---|
| Initial seawater volume | 350 L |

Electrodialysis (ED)

| | |
|---|---|
| Number of electrodialysis | 50 |
| compartments | |
| Brine solution | 20 L of purified |
| | water + NaCl + 0.1N HCl, |
| | so as to obtain a conductivity of |
| | 10 mS/cm and a pH of 1.6 |
| Electrolyte solution | 20 L of purified water + Nitric |
| | acid (HNO3), so as to obtain a |
| | conductivity of 20 mS/cm |

Flow rate

| | |
|---|---|
| Product | 1000 I/h |
| Brine | 1000 I/h |
| Electrolyte | 360 I/h |
| Electrodialysis voltage (ED) | 45 V |
| Evolution of the | 54 to 17 mS/cm |
| conductivity for ED | |
| Evolution of the pH for ED | 7.4 to 3.7 |
| Amount of water lost | 30 I, i.e. a volume yield of 91% |
| during the ED | |

Nanofiltration (NF)

| | |
|---|---|
| Evolution of conductivity for NF | 17 to 21 mS/cm |
| Evolution of the pH for NF | Almost constant: from 3.8 to 4.1 |
| Final volume obtained | 192 I, i.e. a yield of 60% |

TABLE 3
Characteristics of the composition derived from seawater obtained

| | | |
|---|---|---|
| Solution salinity after NF | 12 | g/l |
| Sodium concentration | 1610 | mg/l |
| Magnesium concentration | 1770 | mg/l |
| Calcium concentration | 575 | mg/l |
| Potassium concentration | 51 | mg/l |

### Example 2: Immediate Soothing Effect of the Composition Derived from Seawater

Material and Method:

The immediate soothing effect of the composition derived from seawater is measured after standardized application of a 10% lactic acid solution versus physiological serum on the nasolabial folds of 22 volunteers with sensitive skin on the face (Stinging Test Method).

The composition, derived from seawater, is tested pure and sprayed by mist on the skin of the face.

This test is based on the object's objective self-assessment of the tingling sensation felt on the right and left, after application of the solutions, every minute, for several minutes.

The tingling sensation is rated from 1 to 3:

0=nothing

1=slight tingling

2=moderate tingling

3=intense tingling

and the sum of the two scores, at 30 sec, 5 minutes and 15 minutes, is calculated.

A sum of 3 or greater on the side where lactic acid was applied defines the object as “STINGER” and the object may be included.

The soothing effect of a product is determined on objects initially designated “STINGER”.

If the sum of the side where the lactic acid was applied is less than 3 after application of the product, then the soothing effect is confirmed.

Results

A significant decrease in the intensity of tingling on the nasolabial fold treated with the product was obtained, namely a decrease in:
32% 30 sec after application of the product, in 45% of objects,
57% 5 min after application of the product, in 82% of objects, and
23% 15 min after application of the product, in 86% of objects.

A significant decrease in the duration of tingling on the nasolabial fold between the area with the product and the area without the product was observed in favor of the area with the product by 45% on average. This effect was observed in 68% of objects.

The mist consisting of the composition derived from seawater can therefore be described as immediate soothing.

### Example 3: Hydrating Effect of the Composition Derived from Seawater

Material and Method

Hydration kinetics is a technique based on measuring the hydration index of the surface layers of the skin.

For this, we use corneometry, a method based on the existing relationship between the electrical properties of tissues and their water content. A measurement is taken on a control area and on an area where the product has been applied in a unique way. Several measurements will be carried out (TO, 1 h, . . . , 8 h).

10 female adult volunteers, aged 18 to 70 years old and with dry skin on the legs (corneometric values less than or equal to 50 a.u.), are included in the study. During this, 0.07 ml of mist consisting of the composition derived from seawater was applied to the legs of the volunteers over an area of 35 cm².

Results

The mist consisting of the composition derived from seawater exhibited a statistically significant hydrating activity of the superficial layers of the epidermis 8 hours after application, with a maximum hydration gain of 19.6%.

### Example 4: Restructuring Effect of the Composition Derived from Seawater

Material and Method

The restructuring effect of the seawater composition is tested in a model of human skin explants.

As in Example 2, the composition derived from seawater is formulated as a gel, at 3 different concentrations (2%, 10% or 50%, the percentage being expressed in g/100 g of composition).

The skin explants are supplied by the company Bioprédic International (Rennes) and kept alive in the laboratory in an appropriate culture medium. The explants are pretreated for 24 h with the formulated product or a placebo (not comprising a composition derived from seawater) applied topically, then stimulated or not by irradiation with UVA and UVB rays for 24 h. The product to be tested or the placebo formula is reapplied during stimulation.

Results The restructuring effect of the composition derived from seawater is evaluated by visualizing the effect produced on the barrier function of the skin, in particular on the formation of tight junctions, in particular the claudins 1 and 4. Indeed, those—these participate in maintaining the homeostasis of the stratum corneum by controlling the calcium gradient. When the barrier formed by the tight junctions is altered, for example by UV in the model used, the calcium gradient is disturbed, which leads to an alteration in epidermal differentiation. Tight junctions are intercellular junctional complexes that provide adhesion between keratinocytes in the stratum granulosum.

For claudin 1, its alteration (meshing defect) is observed in the event of UV irradiation (UVA+UVB) (effect visualized on the majority of images of explants in the irradiated control condition, results not shown). Each of the formulas applied topically (2%, 10% or 50%) as well as the placebo makes it possible to obtain an improvement in the altered mesh size following UVs, compared to the irradiated control. The formula comprising 50% of the composition derived from seawater allows a better restoration of the alterations caused by UV irradiation than that observed with the placebo.

For claudin 4, irradiation also alters the expression of this claudin (mesh defect, finer areas), and therefore modulates the integrity of this cell junction. The topical application of the formulations tested makes it possible to restore the destructuring of the mesh observed in the presence of irradiation. As before, the formula comprising 50% of the composition derived from seawater has a better effect than the placebo.

As before, the effect of the composition derived from seawater at the concentrations tested is possibly masked by the effect of freezing.

In conclusion, the composition derived from seawater has an interesting effect on the two proteins studied, allowing the restoration of the expression of claudins 1 and 4 altered by UV irradiation, in particular at a concentration of 50%. The composition derived from seawater therefore makes it possible to protect the skin from alterations in proteins participating in the maintenance of tissue integrity, of homeostasis and of the barrier function of the epidermis, and therefore of maintaining the barrier function of the skin. ‘epidermis.

### Example 5: Examples of Cosmetic Composition Based List on a Composition Derived from Seawater

No-rinse cleaning water

| | | Type of |
|---|---|---|
| INCI list | % | ingredient |
| COMPOSITION DERIVED | 50.000000 | Active ingredient |
| FROM SEAWATER | | |
| PURIFIED WATER | 37.680000 | solvent |
| °1,3-Propylene Glycol | 5.000000 | solvent |
| GLYCERINE | 3.000000 | Humectant |
| ° Polyoxyethylene (20) | 3.000000 | Surfactant |
| Sorbitan Monolaurate | | |
| α-D-glucopyranose | 0.500000 | Soothing active |
| monomers | | agent |
| PERFUME | 0.300000 | Perfume |
| L-Glutamic acid, N, N-Bis | 0.300000 | Chelating agent |
| (Carboxymethyl)-, | | |
| Tetrasodium salt | | |
| Sodium benzoate | 0.350000 | Preservative |
| °1,2-Octanediol | 0.100000 | solvent |
| CITRIC ACID | 0.07000 | Buffering agent |

Body and Hair Cleansing Gel

| | | Type of |
|---|---|---|
| INCI list | % | ingredient |
| PURIFIED WATER | 69.4995 | solvent |
| PURIFIED WATER | 10.000000 | Active ingredient |
| °Cocamidopropyl Dimethyl Glycine | 6.000000 | Surfactant |
| GLYCERINE | 5.000000 | Humectant |
| COCO-GLUCOSIDE | 5.000000 | Surfactant |
| Glycerine Monooleate | 1.000000 | emollient |
| ACRYLATES/C10-30 ALKYL | 0.800000 | Viscosity control agent |
| ACRYLATE CROSSPOLYMER | | |
| α-D-glucopyranose monomers | 0.500000 | Active soothing agent |
| PERFUME | 0.500000 | perfume |
| Sodium benzoate | 0.450000 | preservative |
| soda | 0.300000 | Buffering agent |
| Allantoin | 0.200000 | Active ingredient |
| | | (soothing agent) |
| Benzoic acid | 0.200000 | preservative |
| Cellulose, 2-Hydroxyethyl | 0.200000 | Antistatic agent |
| 2-Hydroxy-3-(Trimethylammonio) | | |
| Propyl Ether, Chloride | | |
| L-Glutamic acid, N, N-Bis | 0.200000 | Chelating agent |
| (Carboxymethyl) -, Tetrasodium salt | | |
| Citric Acid | 0.150000 | Buffering agent |
| Vitamin E | 0.000500 | Antioxidant agent |

Moisturizer

| | | Type of |
|---|---|---|
| INCI list | % | ingredient |
| COMPOSITION DERIVED | 50.000000 | Active ingredient |
| FROM SEAWATER | | |
| Purified water | 17.050000 | solvent |
| Octanoic/Decanoic | 7.000000 | Emollient |
| Acid Triglyceride | | |
| 1,3-Propylene Glycol | 5.000000 | solvent |
| Polyethylene Glycol 1000 | 3.000000 | emulsifier |
| Cetyl/Stearyl Ether | | |
| Glycerine | 5.000000 | Humectant |
| C10-18 TRIGLYCERIDES | 3.000000 | Emollient |
| POLYACRYLATE | 2.000000 | Viscosity control |
| CROSSPOLYMER-6 | | agent |
| OLEYL ERUCATE | 2.00000 | Emollient |
| Shea Butter | 2.000000 | Active ingredient |
| | | (skin conditioning |
| | | agent) |
| Sweet almond oil | 2.000000 | Active ingredient |
| | | (skin conditioning |
| | | agent) |
| PERFUME | 0.600000 | perfume |
| α-D-glucopyranose | 0.500000 | Active soothing agent |
| monomers | | |
| SQUALANE | 0.255000 | Active ingredient |
| | | (skin conditioning |
| | | agent) |
| XANTHAN GUM | 0.200000 | Stabilizing agent |
| | | for an emulsion |
| L-Glutamic acid, N, N-Bis | 0.150000 | Chelating agent |
| (Carboxymethyl) -, | | |
| Tetrasodium salt | | |
| Isopropyl Methylphenol | 0.100000 | preservative |
| Polyoxyethylene (20) | 0.055000 | Surfactant |
| Sorbitan Monostearate | | |
| vitamin E | 0.052000 | Antioxidant agent |
| Citric Acid | 0.023000 | Buffering agent |
| Isorbitan stearate | 0.015000 | Emulsifying agent |

Intimate hygiene cleansing gel

| | | Type of |
|---|---|---|
| INCI list | % | ingredient |
| Purified water | 71.64600 | solvent |
| COMPOSITION DERIVED | 10.000000 | Active ingredient |
| FROM SEAWATER | | |
| Cocamidopropyl Dimethyl Glycine | 6.000000 | Active ingredient |
| | | (cleaning agent) |
| Glycerine | 4.000000 | solvent |
| Polyethylene Glycol (7) | 2.000000 | Surfactant |
| Glyceryl Monococoate | | |
| Polyoxyethylene (20) | 1.500000 | Surfactant |
| Sorbitan Monolaurate | | |
| Sodium N-Lauroyl-L-glutamate | 1.240000 | Surfactant |
| ACRYLATES/C10-30 ALKYL | 1.200000 | Viscosity agent |
| ACRYLATE CROSSPOLYMER | | |
| Polyethylene Glycol (120) | 0.596000 | Viscosity agent |
| Methyl Glucose Dioleate | | |
| SODA | 0.400000 | Buffering agent |
| Pro-vitamin B5 | 0.375000 | Active ingredient |
| | | (skin conditioning |
| | | agent) |
| PERFUME | 0.300000 | perfume |
| Sodium Benzoate | 0.350000 | Preservative |
| Sodium N-Lauroyl-L-glutamate | 0.200000 | Chelating agent |
| Lactic acid | 0.090000 | Buffering agent |

Superfat soap

| | | Type of |
|---|---|---|
| INCI list | % | ingredient |
| SODIUM PALMATE | 64.333600 | Surfactant |
| SODIUM PALM KERNELATE | 15.576000 | Surfactant |
| Purified water | 13.05800 | solvent |
| COMPOSITION DERIVED | 2.000000 | Active ingredient |
| FROM SEAWATER | | |
| PERFUME | 1.500000 | perfume |
| Shea Butter | 1.000000 | Active ingredient |
| | | (skin conditioning |
| | | agent) |
| PALM ACID | 0.660800 | Free fatty acids |
| | | (saponification |
| | | residues) |
| Sodium chloride | 0.566400 | Viscosity control |
| | | agent |
| Titanium dioxide | 0.500000 | colorant |
| GLYCERINE | 0.472000 | Humectant |
| PALM KERNEL ACID | 0.188800 | Free fatty acids |
| | | (saponification |
| | | residues) |
| PROPYLENE GLYCOL | 0.050000 | solvent |
| Sodium Edetate | 0.047200 | Chelating agent |
| Tetrasodium | 0.047200 | Chelating agent |
| 1-Hydroxyethane-1, | | |
| 1-Diphosphonate | | |

Mist

| | | |
|---|---|---|
| COMPOSITION DERIVED | 100.000000 | Active ingredient |
| FROM SEAWATER | | |

What is claimed is:

1. A composition derived from seawater, wherein said composition comprises potassium at a concentration less than or equal to 100 mg/l and magnesium at a concentration greater than or equal to 1600 mg/l.

2. The composition derived from seawater according to claim 1, wherein said composition comprises magnesium at a concentration of 1600 mg/l to 3000 mg/l.

3. The composition derived from seawater according to claim 1, wherein said composition is hypertonic.

4. The composition derived from seawater according to claim 1, wherein said composition comprises:
sodium at a concentration less than or equal to 1800 mg/l, and/or
calcium at a concentration greater than or equal to 460 mg/l.

5. A device comprising the composition derived from seawater according to claim 1.

6. A composition comprising the composition derived from seawater according to claim 1.

7. The composition according to claim 6, wherein said composition is a cosmetic composition.

8. A method for obtaining a composition derived from seawater according to claim 1, wherein said method comprises:
a step of selective electrodialysis of a sample of seawater, optionally filtered, until a solution is obtained with a conductivity of 15 mS/cm to 18 mS/cm, and
a step of nanofiltration of said solution.

9. (canceled)

10. (canceled)

11. A method for preparing a cosmetic composition according to claim 7, wherein said method comprises a step of mixing a composition derived from seawater with at least one other cosmetic ingredient,
wherein said composition derived from seawater comprises potassium at a concentration less than or equal to 100 mg/l and magnesium at a concentration greater than or equal to 1600 mg/l or is obtained by the method comprising:
a step of selective electrodialysis of a sample of seawater, optionally filtered, until a solution is obtained with a conductivity of 15 mS/cm to 18 mS/cm, and
a step of nanofiltration of said solution.

12. A cosmetic treatment method, wherein said method comprises the topical application of a cosmetic composition according to claim 7.

13. A cosmetic treatment method, wherein said method comprises the topical application of a cosmetic composition obtained by the method according to claim 11.
